(19) United States
(12) Patent Application Publication
(10) Pub. No.: US 2001/0055761 A1
(43) Pub. Date: Dec. 27, 2001

(54) SMALL SCALE DNA SYNTHESIS USING POLYMERIC SOLID SUPPORT WITH FUNCTIONALIZED REGIONS

(75) Inventors: ROY H. KANEMOTO, PALO ALTO, CA (US); MICHAEL G. M. PERBOST, CUPERTINO, CA (US)

Correspondence Address: AGILENT TECHNOLOGIES, INC., INTELLECTUAL PROPERTY ADMINISTRATION, LEGAL DEPT., P.O. BOX 7599, M/S DL429, LOVELAND, CO, 80537-0599, US

(73) Assignee: Agilent Technologies

(21) Appl. No.: 09/429,054

(22) Filed: Oct. 29, 1999

(51) Int. Cl. C12Q 1/68; C07H 21/00; C07H 21/02; C07H 21/04; B32B 5/02; B01L 11/00; B32B 27/04; G01N 33/543; B32B 27/12; G05B 17/00; C08F 2/00

(52) U.S. Cl. 435/6; 422/131; 422/101; 422/116; 436/518

(57) ABSTRACT

The invention relates to method and apparatus for synthesis of polymers, and specifically teaches the use of polymeric sheets suitable for the synthesis of small quantities of oligonucleotides such as DNA.

The polymeric sheets may be formed from a variety of materials, and wells or walled chambers formed by drilling or molding. The surfaces designated for synthesis product attachment sites are suitably functionalized. The well or walled chambers are of predetermined depth, corresponding to the relative amount of product desired to be synthesized per well. Sheets placed on an X-Y platform are amenable to automated synthesis protocols. After synthesis, collection of product may be immediate, or sheets may be stored and collection from predetermined wells may be made as desired. Many oligonuclotidess may be simultaneously synthesized, and quantities suitably controlled to ensure efficient production of desired product.

### BACKGROUND OF THE INVENTION

1. Field of Invention

This invention relates to support substrates for synthesis, and most particularly to the use of a multilayer polymeric substrate for synthesis of organic compounds, most specifically for nucleic acid synthesis.

2. Background of the Invention

Articles and publications set forth in this patent disclosure are presented for the information contained therein; none of the information is admitted to be statutory “prior art” and we reserve the right to establish prior inventorship with respect to any such information.

While well established protocols exist for synthesis of many heteropolymers, most biopolymers cannot be stored once activated, and there is currently no practical method for synthesizing small quantities of heteropolymers at a reasonable cost.

A well known chemical procedure for the synthesis of nucleic acids, including RNA and DNA, is commonly referred to as the “phosphoramidite methodology.” See U.S. Pat. No. 4,415,732: McBride, L. and Caruthers, M. Tetrahedron Letters 24:245-248 (1983); and Sinha, N. et al. Nuc. Acids Res. 12:4539-4557 (1984) all incorporated herein by reference. Commercially available oligonucleotide synthesizers use the phosphoramidite methodology and most commonly oligonucleotides are “grown” on a support material, or “solid support”.

Oligonucleotides—synthetic strands of DNA and RNA—are important owing to the wide variety of applications in which they may be exploited. Oligonucleotides are useful in biological studies ranging from genetic engineering and recombinant DNA to primers for amplification (e.g. Polymerase Chain Reaction—PCR) and studies of molecular interaction.

Proteins, as well as nucleic acids, have been chemically synthesized. Known as solid phase peptide synthesis, procedures for the synthesis of linear amino acid sequences were introduced in 1963. See generally, Barany, G. and Merrified, R. B. (1980) in The Peptides, 2:1-284. Gross, E. and Meienhofer, J. Eds. Academic Press, New York.

Oligosaccharides, too, have been synthesized using solid support. See Douglas, S. P. et al J. Am Chem Soc 113:5095-5097 (1991). And see Rudemacher, T. W. et al. “Glycobiology” Ann. Rev. Biochem. 57:785-838 (1988).

A variety of support materials are used in the synthesis of nucleic acids, proteins, and oligosaccharides.

Synthesis of DNA has been done using Controlled Pore Glass (CPG) for many years, and CPG is a well-established solid synthesis medium. See, for example, U.S. Pat. No. 4,458,066. Nonetheless, CPG has limitations. Contaminants, silica and polymeric siloxanes, are released during cleavage and deprotection of oligo nucleotides. Moreover, the silane coupling chemistry to funtionalize the inorganic surface of CPG beads is complex, and contributes to the variance in substitution levels from batch to batch. Beaded materials, whether organic or silica-based, present difficulty arising from the time required for diffusion of reagents and washing of solvents in and out of pores. For DNA synthesis, the diffusional requirement severely limits the number of synthesis cycles that may be completed in any fixed time period, although the reactions themselves are quite rapid. Beaded supports also introduce particulates into the fluidic systems of automated synthesizers. This is a common problem with beaded supports and automated synthesizer models commercially available from such companies as Beckman Instruments, Millipore, and Perkin Elmer Applied Biosystems.

To better underscore the complexity of the automated oligonucleotide synthesis apparatus as currently employed, we here set forth a few basic points relevant to DNA and RNA sequencing. Each DNA or RNA molecule is a linear biopolymer consisting of a string or sequence of nucleotides that encode the genetic information for that DNA or RNA molecule. Each nucleotide monomer consists of a nucleoside (a nitrogenous base linked to a pentose sugar) and a phosphate group. Nucleotides are identified according to the nitrogenous base, i.e. adenosine, cytosine, guanine, and thymine or uracil.

Controlled Pore Glass (CPG) is a common synthesis support for nucleotide chain and is typically used “as-is” or embedded in Teflon. The CPG is loaded into a plastic column that serves as the reaction vessel. The CPG column selected depends on the amount of DNA to be synthesized and the oligonucleotide sequence. The quantity of CPG in the column is related to the amount of DNA synthesized. With regard to the impact of the oligoucleotide sequence upon the selection of the CPG column, current DNA synthesis instruments use CPG with the first nucleotide pre-attached, i.e. there are four different types of CPG, one for each nucleotide. Therefore the first base in the oligonucleotide sequence dictated the selection of the CPG column. Subsequent synthesis cycles build on the first base linked to the CPG to yield the desired oligonucleotide. While the quantity of oligonucleotide synthesized can be customized based upon the CPG column even the smallest scale synthesis produces quantities of an oligonucleotide that in many cases far exceeds the amount that is needed. In addition, the use of oligonucleotides in biotechnology research is largely an empirical activity where screening of a large number of different oligonucleotides is typically done. The result is additional costs in the excessive preparation, handling and storage of unused or wasted materials. The need for small scale synthesis of oligonucleotides and other biopolymers has gone largely unmet.

In addition to solid support for synthesis protocols, membrane supports have been used. Polymeric membranes have been considered for an alternative to CPG for nucleic acid synthesis. (see Innovation and Perspectives in Solid Phase Synthesis, Peptides, Proteins and Nucleic Acids, ch 21 pp 157-162, 1994, Ed. Roger Epton); see also U.S. Pat. No. 4,923,901. Once formed, a membrane can be chemically functionalized for use in nucleic acid synthesis. In addition to the attachment of a functional group to the membrane, the use of a linker or spacer group attached to the membrane may be used to minimize steric hindrance between the membrane and the synthesized chain.

Surface activated polymers have been demonstrated for use in synthesis natural and modified nucleic acids, proteins on several solid supports mediums.

Increasingly, there is a need for many oligonucleotide sequences simultaneously available, each in small quantities. Examples include identification of primers for PCR, use in multiplex PCR for expression profiling on a DNA array, sample preparation for DNA arrays in general, and whole oligonucleotides deposition on DNA arrays. DNA arrays are synthesized by spotting already made oligonucleotides, instead of a synthesis base after bases. For the preparation of whole oligo arrays, current small scale synthesis yields enough material for thousands of arrays, more than what is needed for preliminary experiments and screening. What is needed is a means for parallel production of oligonucleotides. What is also needed is a method for synthesizing a number of different oligonucleotides in parallel and at high throughput. Also needed is a method is simpler and faster than CPG methods commonly used, and that the reagent usage is efficient, thereby reducing not only the cost but the environmental impact. Further needed is a method of biopolymer synthesis that permits synthesis in small batches. Also needed is a support for synthesis that meets all the needs set forth herein.

### BRIEF SUMMARY OF THE INVENTION

The method taught is an improved method and material adapted for synthesizing oligonucleotides, such method especially adaptable for small scale synthesis. The method provides for sequentially coupling bases to form the desired oligonucleotide, wherein an improvement includes sequentially coupling bases to dimensionally specific, walled, functionalized regions within a polymeric support material. The walled chambers in the support material may be formed by molding, or drilling, or any means that produces walled chambers of predetermined dimensions.

The method for oligonucleotide synthesis adapted for small quantities of oligonucleotide, includes the steps of selecting a polymeric material in which there have been formed walled chambers of predetermined size; functionalizing said walled chambers sufficient to support oligonucleotide synthesis; synthesizing oligonucleotide within said walled chambers. The oligonucleotides may then be harvested, or stored for later harvesting. In the embodiment in which the polymeric material is initially fairly thick or block-like, it may be further sliced into sheets of predetermined thickness and the sheets and the walled chambers therein then addressably oriented on an X-Y platform. Phosphoramidite solution may be delivered into pre-selected chambers; following the phosphoramidite reaction, the chambers are exposed to the next solution in the synthesis process, whether flush or reagent, repeating these two steps until the synthesis is complete. Harvesting is performed by introduction of ammonia into the chamber from which oligonucleotide is to be harvested. Sheets may be stored with unharvested oligonucleotides. The sheets may be further cut into sections and the selected chambers harvested as desired.

### BRIEF DESCRIPTION OF THE DRAWINGS

FIG. 1 is a block diagram flow chart depicting the inventive method.

### DETAILED DESCRIPTION OF THE PREFERRED EMBODIMENT

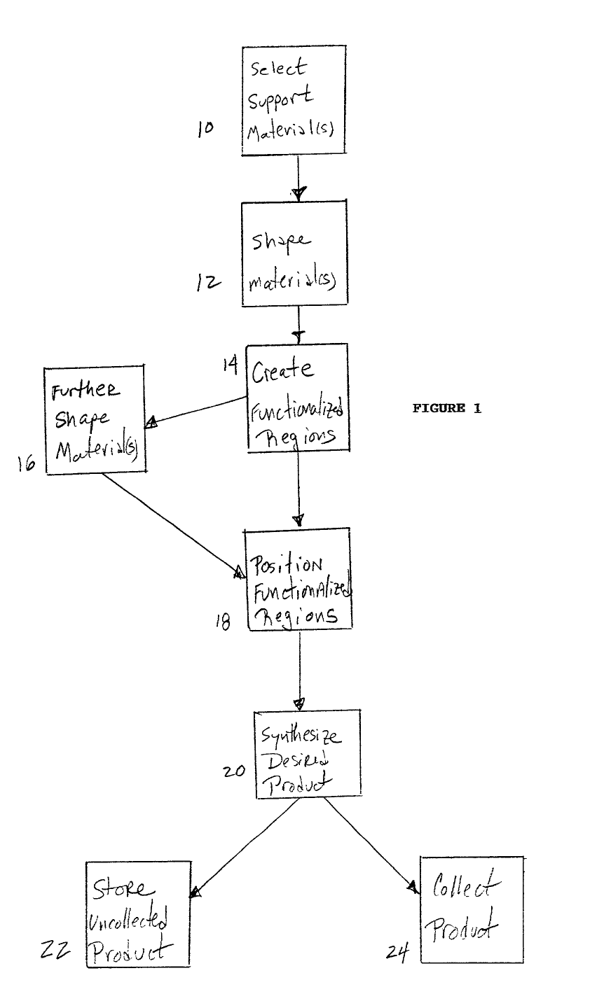

FIG. 1 illustrates, in the form of a flowchart, the inventive method. Initially, a suitable support material is selected 10. The material may be any polymer suitably uniform in porosity, has sufficient amine content, and sufficiently flexible to undergo any attendant manipulations without losing integrity. Examples of suitable selected materials include nylon, polypropylene, polyester, polytetrafluoroethylene, polystyrene, polycarbonate, and nitrocellulose. Other materials may serve, depending on the design of the investigator. In consideration of some designs, for example, a coated metal, in particular gold or platinum may be selected.

According to the design of the investigator, a piece of said support material is selected 12, which, for the purposes of this disclosure, shall be termed a block but the word block shall mean for the purpose of this disclosure any desired shape, said block being or shaped into being of predetermined dimensions; and into said block a series of impressions, wells or chambers, by drilling, molding or other means, are made 14. This operation is performed so as to create walled chambers 16 or wells in the block of predetermined dimensions. The wall of each well extending for approximately 1-3 mm into or entirely through the block. The orientation of each well in the preferred embodiment is about perpendicular to the surface, although it is conceivable that another orientation may be preferred. In alternate embodiments, the walled chambers or wells may be the result of a molding process, rather than of drilling, or some combination of processes in the event that, for example, a coating is desired.

The surface of each well is left rough to maximize the surface area. The free amines in the inner surface of the well are functionalized 14 by any suitable method with a universal type of linker for the oligonucleotide or other species of polymer to be synthesized.

In the preferred embodiment, the linker is universal for DNA synthesis. The amine content of the polymer selected as the support material provides that each walled chamber or well have a uniform loading. If it is desirable to increase the amine content, the amines in the selected substrate material may be reacted with a soluble amine-containing polymer such as polyethyleneimine (PEI). In alternate embodiments, the polymeric material contains free hydroxyls, and an analogous functionalization and linking process is used.

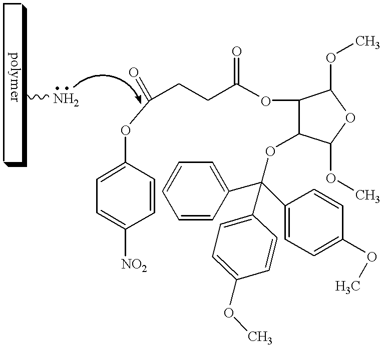

After the wells have been functionalized 14, the block may be further shaped, by means of slicing 16 into two or more sheets, each sheet containing all, or some predetermined subset of, the original block cross section. To practice the invention, it is not required that the block be sliced. However, in the preferred embodiment, sheets of solid support material are formed thusly, with the series of addressable wells therein. The thickness of the sheets may be varied, as different yields may be expected from wells of different depths.

Singly, each sheet is then addressably placed upon an X-Y platform 18 such as a stage or other conveyor oriented so that each walled chamber is addressable. At this stage, synthesis of the desired oligonucleotide 20 then commences. As the steps of synthesis are well known, generalized synthesis protocol steps are set forth briefly here for the purpose of illustration. The required phosphoramidite solution is delivered to each well using a suitable method such as pulse delivery (ex. IVEK pump, valve jet), or methods employed in ink jetting. After the phosphoramidite reaction, the sheet and the wells therein are flushed with or dipped into the next reagent. The delivery, reaction, and flushing steps are repeated for the synthesis cycles required.

Following synthesis, ammonia, or an equivalent cleaving agent is delivered to such wells from which the synthesized oligonucleotides are to be collected, and the collection performed. Un-ammoniated or un-cleaved sheets or wells may be transported and stored for a period of time 22, and oligonucleotide collection being done when desired. The amount of oligonucleotide synthesized per well is a function of the diameter of the well and the depth of the well (that is to say, the thickness of the sheet).

The sheets provide the option of selecting wells for collection, cutting selected wells apart from other wells which are not to be harvested just then, and selectively harvesting the preselected wells. It is worth setting forth here some further elaboration on the universal support for DNA synthesis as taught in the preferred embodiment.

In DNA synthesis, four varieties of solid supports are commonly used—one for each base, A, T, G, and C according to which base is the 3′end of the nucleotide. In the preferred embodiment, the preferred solid support is that which is functionalized with a 3′-dimethoxytrityl ribose, without any nucleobase. The deblocking of this ribose in acidic solution allows DNA synthesis, and the final deprotection reaction generates a RNA type 3′ end that is cleaved from the oligonucleotide under base conditions. Such a solid support is termed herein a universal solid support. If the support is not a solid but a membrane, paper (cellulose) or plastic sheet, the support may not be specifically functionalized with A or T or G or C. In this case, the universal solid support is used, taking advantage of the instability of RNA in basic condition. The universal solid support is functionalized with a ribose, without a nucleobase. Moreover, the functionalization is the reverse of the most typical orientation. Typically on a ribose, the nucleobase would be in the 1 position and the support connection would occur in the 3 position, or 3′ end. The cis diol (alcohol in positions 2′ and 3′) is protected on one alcohol with a dimethoxytrityl and on the other with a succinate linker connected to the polymeric sheet.

The first step of the synthesis is a removal of the dimethoxytrityl group in acidic condition (termed “deblocking”) then introduction of an activated phosphoramidite on the deprotected alcohol.

In this manner, a 3′-3′ or, depending on the location of the dimethoxytrityl group) a 3-2 link is formed rather than a 3′-5′.

When the synthesis is completed, the solid support is soaked in or otherwise exposed to a basic solution (ammonia or ammonia/methylamine or ethanolamine) with the result being the removal of the protecting group on the phosphate groups, on the nucleobase, and cleavage from the support (termed, “deprotection” or “final deprotection”). Now the oligonucleotide if no longer anchored to the support medium, but remaining on the end of the oligonucleotide is the original sugar—the ribose. The 2′ alcoholate will attack the phosphorous, leading to a cleavage of an oligonucleotide-O-P bond (cleavage between the P and O); in other words, the phosphate will stay on the ribose and the oligonucleotide is “free”.

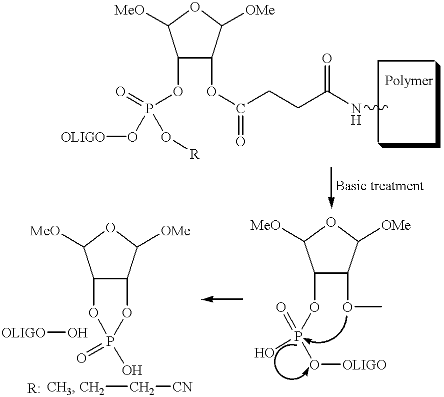

While the foregoing has been described in considerable detail and in terms of preferred embodiments, these are not to be construed as limitations on the disclosure or claims to follow. Modifications and changes that are within the purview of those skilled in the art are intended to fall within the scope of the following claims.

We claim:

1. In a method for synthesizing oligonucleotides attached to a solid support, said method comprising sequentially coupling bases to form said oligonucleotide, wherein the improvement comprises sequentially coupling bases to dimensionally specific, functionalized regions within a polymeric support material.

2. In a method as in claim 1, further including the improvement of synthesized oligonucleotides contained within regions within a polymeric support material, where said support material may be stored and such synthesized oligonucleotides may be selectively collected by separating a portion of the support material from the remainder.

3. A method for oligonucleotide synthesis adapted for producing small quantities of oligonucleotide, comprising the steps:
Selecting a polymeric material in which there have been formed walled chambers of predetermined size;
Functionalizing said walled chambers sufficient to support oligonucleotide synthesis;
Synthesizing oligonucleotide within said walled chambers;
Harvesting said oligonucleotide from predetermined chambers.

4. A method as in claim 3, further comprising the steps of slicing the polymeric material into sheets of predetermined thickness and addressably orienting said sheets on an X-Y platform.

5. A method as in claim 4, further comprising the steps of delivering phosphoramidite solution into pre-selected chambers; following the phosphoramidite reaction, the chambers are exposed to the next solution in the synthesis process, whether flush or reagent, repeating these two steps until the synthesis is complete.

6. A method as in claim 3 wherein the harvesting is performed by introduction of a cleaving reagent into the chamber from which oligonucleotide is to be harvested.

7. A method as in claim 3 wherein the polymeric material containing the oligonucleotide-containing chambers are stored for some time prior to harvesting.

8. A polymeric material adaptable for small scale oligonucleotide synthesis wherein said material is of predetermined dimensions and contains walled chambers suitable for creating functionalized regions for the synthesis of oligonucleotides.

9. A material as in claim 8 wherein said material may be sliced to predetermined thickness in a predetermined orientation to the center of any chamber, such predetermination of thickness having the effect of selecting the amount of product that may be synthesized in such chamber by altering the wall space in any chamber.

10. A material as in claim 8 wherein products of synthesis may be stored prior to collection, and wherein the material may be cut or otherwise divided into sub-sections for collection.
